(19) United States
(12) Patent Application Publication
(10) Pub. No.: US 2022/0280096 A1
(43) Pub. Date: Sep. 8, 2022

(54) SSVEP-BASED ATTENTION EVALUATION METHOD, TRAINING METHOD, AND BRAIN-COMPUTER INTERFACE

(71) Applicant: HANGZHOU MINDANGEL LTD., Hangzhou (CN)

(72) Inventor: Xing SONG, Hangzhou (CN)

(73) Assignee: HANGZHOU MINDANGEL LTD., Hangzhou (CN)

(21) Appl. No.: 17/637,110

(22) PCT Filed: Dec. 12, 2019
(86) PCT No.: PCT/CN2020/101268

§ 371 Date: Feb. 22, 2022

(30) Foreign Application Priority Data
Aug. 23, 2019 (CN) 201910783497.1

(51) Int. Cl. A61B 5/378; A61B 5/16; A61B 5/00

(52) U.S. Cl. CPC A61B 5/378; A61B 5/168; A61B 5/7425; A61B 5/72

(57) ABSTRACT

An SSVEP-based attention evaluation method, a training method, and a brain-computer interface are provided. The vision of a subject (3) is stimulated by means of displaying test image(s) (2), the brain waves of the subject (3) are collected, the SSVEP in the brain waves is extracted, and the amplitude features of the SSVEP are used to represent the degree of attention of the subject (3). The present method achieves quantification of the degree of attention and increases the accuracy and usability of attention evaluation, increases the dimension of information that EEG signals can reflect, increases the usability of the brain-computer interface, and widens the motion control function of the brain-computer interface.

### TECHNICAL FIELD

The present invention relates to the technical field of EEG recognition, and in particular, to an SSVEP-based attention evaluation method, a training method, and a Brain-Computer Interface (BCI).

### BACKGROUND

A BCI is a direct connection passage between human or animal brains and external devices. At present, the BCI mainly collects and analyzes EEG signals under different states, and then uses certain technical means to establish a direct communication and control channel between the human brain and computer or other electronic devices, so that a person can directly control own brain activities to control external devices, without the need for language and limb motions. Attention refers to the ability of a person's mental activity to point to and concentrate on something. Attention is an aspect of the study of EEG signals, which can help to understand the level of attention concentration of a person to some extent. At present, there are mainly behavioral observation methods or brain wave band energy methods to judge the degree of attention concentration. The former can only make qualitative analysis, while the latter can only make rough attention level division. Meanwhile, there are problems such as large delay, inaccurate classification, and external interference.

SSVEP, i.e. steady-state visual evoked potential, refers to a continuous response related to a visual stimulation frequency when the brain visual cortex of a person is subjected to a fixed frequency of visual stimulation. SSVEP may be reliably applied to Brain-Computer Interface Systems (BCIs). SSVEP BCIs generally have a higher information transmission rate than BCIs that give other signals, such as P300 and motor imagery. The system and experimental design are simpler, and less training is required. The existing technologies use SSVEP to determine the gaze target of a subject, but do not involve the use of SSVEP of the subject to detect the degree of attention concentration. For example, in Chinese patent CN105302309B, published on Jan. 12, 2018, a method for identifying brain wave instructions based on an SSVEP BCI is provided. Human vision is stimulated by a flicker frequency of a stimulation source, and the stimulation source is encoded and decoded by a method of “instruction code element+feature bit”. The instruction code element is code of an instruction, and the feature bit is located between instruction code elements with a certain number of bits, and used for interval identification. An electrode is used to collect EEG signals generated by stimulation. The collected original EEG signals are filtered and demodulated to finally obtain an instruction code element, thereby obtaining a desired instruction type.

### SUMMARY

The object of the present invention is to solve the technical problem that the existing attention evaluation method cannot quantify the degree of attention, and the present invention proposes an SSVEP-based attention evaluation method, a training method, and a BCI for quantifying the degree of attention.

The object of the present invention is achieved by the following technical solutions:

An SSVEP-based attention evaluation method includes the following steps.

Test image(s) is/are displayed at a frequency f, the vision of a subject is stimulated, and the brain waves of the subject are collected.

An SSVEP related to the frequency f in the brain waves is extracted.

A representation Aₓ of the amplitude features of the SSVEP is used to evaluate the degree of attention of the subject. Aₓ is a combination of the amplitude of the first-harmonic/fundamental frequency f and/or the amplitude of multiple frequency.

Further, a specific calculation formula of Aₓ is as follows:

$${A}_{x} = {\sum }_{i = 1}^{n} {a}_{i} \cdot {M}_{{f}_{i}}$$

where aᵢ is a weighting coefficient, and M_{fi} represents a function of the amplitude of the i^{th} harmonic component in the SSVEP.

Further, a calculation formula of the weighting coefficient aᵢ is as follows:

$${a}_{i} = \{ \begin{matrix}1 , & i is an odd number , & i < 6 \\ 0 , & i is an even number , & i < 6\end{matrix}$$

Further, before an attention evaluation for a new subject, m pre-evaluations are performed, a maximum representation value Aₘₐₓ of the amplitude features of the SSVEP in each pre-evaluation is recorded, a maximum value of the maximum representation values Aₘₐₓ in the m pre-evaluations is taken as a reference value A_{ref} of the evaluation for the subject, the representation of the amplitude features of the SSVEP obtained from a formal evaluation is Aₓ, and then an instantaneous evaluation score for the attention of the subject is:

$$A T = \frac{{A}_{x}}{{A}_{r e f}} \times 100.$$

Further, a final score of each evaluation for the subject is Score=f (AT, t), which is a distribution feature of the instantaneous evaluation score AT for the attention of the subject over time t.

Further, the Score is divided into a short-range average attention score, a medium-range average attention score and a long-range average attention score, and a specific calculation formula is as follows:

Score_{Δt}=∫ₜ_{₀}^{t}^{₀}^{+Δt}ATₓ/Δt

where t₀ is a timing start time, Δt is a time interval, and Δt has different values for a short range, a medium range and a long range.

An SSVEP-based attention training method includes the following steps.

Test image(s) is/are displayed at a frequency f, the vision of a trainee is stimulated, and the brain waves of the trainee are collected.

An SSVEP related to the frequency f in the brain waves is extracted.

A representation of the amplitude features of the SSVEP is used as an instantaneous evaluation result of the attention of the trainee.

The instantaneous evaluation result is fed back to the trainee in real time, so that the trainee adjusts the attention according to the instantaneous evaluation result in real time.

Further, the instantaneous evaluation result is fed back to the trainee in real time during the training process, and a target guidance value at each moment is given at the same time, so that the trainee performs attention adjustment according to the real-time instantaneous evaluation result and the target guidance value.

Further, an attention adjustment training is also included, specifically including the following operations.

The display brightness of the test image(s) is/are increased or decreased in the training, the trainee is guided to perform the attention adjustment training, and the change of brightness is ended when the instantaneous evaluation result returns to the level before the change of brightness or exceeds a set duration.

Further, the test image(s) include(s) a plurality of target images, and at least one of display frequency, color and brightness attributes of the plurality of target images is different.

Further, the degree of coincidence of the attention change of the trainee with the dynamically changing target guidance value is displayed in real time in the training process.

Further, the trainee adjusts the attention according to the target guidance value, and when an error between the instantaneous evaluation result of the attention of the trainee and the target guidance value falls within 5%, the target guidance value is maintained for a period of time t and then changed.

An SSVEP-based BCI includes a visual evoking device for displaying an SSVEP-evoked image, and an EEG collection device worn on the head of a user. The BCI is configured to implement the method of any one of the above.

The SSVEP-based BCI further includes an EEG analyzer. The EEG analyzer is connected to the EEG collection device, and is configured to extract an SSVEP related to a display frequency from the brain waves collected by the EEG collection device, obtain a representation Aₓ of the amplitude features of the SSVEP corresponding to each display frequency, and use a maximum value in the representation and the corresponding frequency as the output of the BCI.

Further, the EEG analyzer distinguishes attention targets of users according to the display frequency corresponding to the maximum value in the representation of the amplitude features of the SSVEP, distinguishes the attention targets by the size of the representation of the amplitude features of the SSVEP if the display frequencies of the target images are the same, and outputs the attention targets.

Further, a lower threshold of the representation value of the amplitude features of the SSVEP is preset, and when the representation value of the amplitude features of the SSVEP is lower than the lower threshold, the EEG analyzer outputs a stop signal or a zero value.

An upper threshold of the representation value of the amplitude features of the SSVEP is preset, and when the representation value of the amplitude features of the SSVEP is higher than the upper threshold, the EEG analyzer outputs the upper threshold.

Further, the EEG analyzer records and outputs a duration for which the instantaneous representation value of the amplitude features of the SSVEP is greater than the lower threshold, the duration is divided into a short duration and a long duration according to a preset duration threshold, and triggering operations corresponding to the short duration and the long duration are output.

Application of an SSVEP-based BCI for controlling the motion of a controlled object is provided. A relationship between a running speed Vₓ of the controlled object and an SSVEP-based amplitude representation value Aₓ satisfies:

$${V}_{x} = \{ \begin{matrix}k \cdot {A}_{x} , & k \cdot {A}_{x} < {V}_{max} \\ {V}_{max} , & k \cdot {A}_{x} \geq {V}_{max}\end{matrix}$$

where k is a weighting coefficient, and Vₘₐₓ is the highest speed at which the controlled object runs.

Further, the weight coefficient k is calculated by the following formula:

$$k = \frac{{V}_{max}}{2 \cdot {A}_{m e a n}}$$

where Aₘₑₐₙ is a mean value of the amplitude representation values of the SSVEP of users over a period of time.

The advantageous effects of the present invention are as follows:

The SSVEP-based attention evaluation method of the present invention achieves fine quantification of the degree of attention concentration and increases the accuracy and usability of attention concentration evaluation, increases the dimension of information that EEG signals can reflect, increases the usability of the brain-computer interface, and widens the motion control function of the brain-computer interface.

### BRIEF DESCRIPTION OF THE DRAWINGS

FIG. 1 is a flow block diagram of an attention evaluation method;

FIG. 2 is a schematic diagram of attention evaluation/training;

FIG. 3 is a schematic diagram of several representation forms of Aₓ;

FIG. 4 is a flow block diagram of an attention training method; and

FIG. 5 is a schematic diagram of a BCI structure.

In the drawings, 1—visual evoking device, 2—test image, 3—subject, 4—EEG collection module, 5—EEG analyzer, 6—output module, 7—SSVEP-evoked image array, 41—headband with EEG electrodes, 42—EEG amplification module, 43—analog-to-digital conversion module.

### DETAILED DESCRIPTION OF THE EMBODIMENTS

Singular items in the application also include the case of plural items.

Objects and effects of the present invention will become more apparent from the following detailed description of the present invention when taken in conjunction with the accompanying drawings and preferred embodiments. It should be understood that specific embodiments described herein are illustrative only and are not intended to limit the present invention.

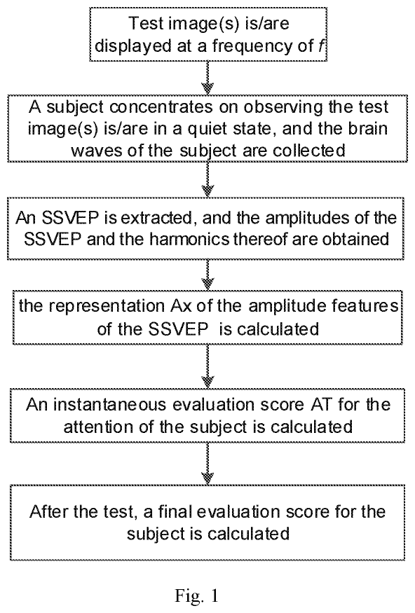

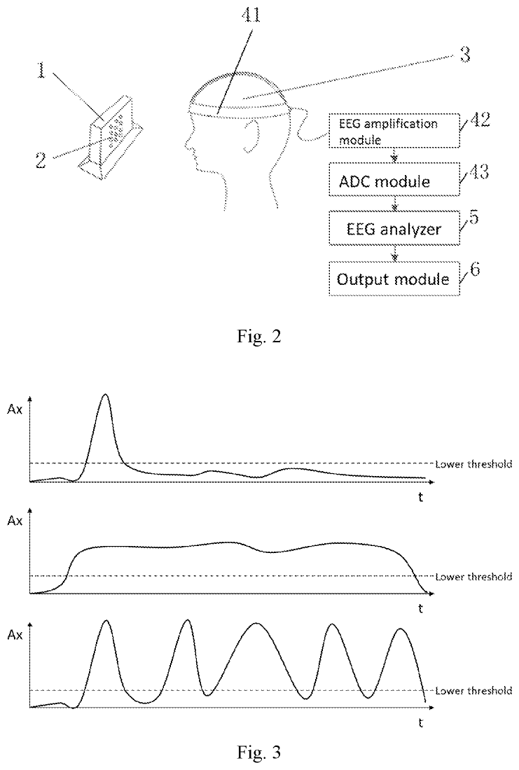

The SSVEP-based attention evaluation method provided by the present invention, as shown in FIGS. 1 and 2, includes the following steps. A vision evoking device 1 displays test image(s) 2 at a frequency of f, and a subject 3 gazes at the vision evoking device 1. The vision evoking device 1 displays several test image(s) 2, and at least one of display frequency f, color and brightness attributes of all the test images 2 is different. According to different evaluation tasks, one or more test images 2 may be selected to be displayed simultaneously. Different frequencies or colors give visual stimuli to the subject 3, different EEG signals may be obtained, and light having a wavelength of 550 nm has the most prominent effect. An EEG collection device 4 includes a headband 41 with electrodes, an EEG amplification module 42 and an analogue-to-digital conversion module 43. The headband 41 with electrodes is worn on the head of the subject 3, the electrodes in the headband 41 are electrically connected to the EEG amplification module 42, and the EEG amplification module 42 is electrically connected to the analogue-to-digital conversion module 43. After collecting the brain waves of the subject 3 by the EEG collection device 4, the EEG collection device 4 is electrically connected to an EEG analyzer 5. The EEG collection device 4 sends the collected EEG signals to the EEG analyzer 5. The EEG analyzer 5 extracts SSVEP signals from the EEG signals. The EEG analyzer 5 obtains frequency and amplitude characteristics of the SSVEP after analyzing the SSVEP using a frequency and amplitude extraction method. By means of the extraction of the SSVEP, the component of the frequency f may be extracted, any harmonic of the frequency f may also be extracted, and a wave composed of the frequency f and several harmonics may also be used as the SSVEP, so as to evaluate the degree of instantaneous attention concentration of the subject through the representation Aₓ of the amplitude features of the SSVEP. The EEG analyzer 5 is electrically connected to an output module 6, and the EEG analyzer 5 sends the analyzed frequency and amplitude characteristics of the SSVEP and the representation Aₓ of the amplitude features of the SSVEP to the output module 6.

FIG. 3 shows several representation forms of Aₓ.

As an implementation mode, a calculation formula of Aₓ is as follows:

$${A}_{x} = {\sum }_{i = 1}^{n} {a}_{i} \cdot {M}_{{f}_{i}}$$

where at is a weighting coefficient, and M_{fi} represents a function of the amplitude of the i^{th} harmonic component in the SSVEP. The weighted sum may be replaced by other operations, such as the product of the components. As an implementation mode,

$${a}_{i} = \{ \begin{matrix}1 , & i is an odd number , & i < 6 \\ 0 , & i is an even number , & i < 6\end{matrix}$$

M_{fi} may be expressed by the amplitude of the i^{th} harmonic component in the SSVEP, or the extraction of the square of the amplitude.

According to the SSVEP extraction method, the components of the target frequency may be directly extracted by narrow-band filtering. Fourier transformation or AR models may also be used. The Fourier transformation method is preferably short-time Fourier transformation, and a window function with an overlapping region is used to perform the Fourier transformation by periodically sliding so as to output a transformation result in real time. The extraction of the frequency using the filtered and noise-reduced pre-processed brain waves will have a better effect.

Before an attention evaluation for a new subject, m pre-evaluations are performed, a maximum representation value Aₘₐₓ of the amplitude features of the SSVEP in each pre-evaluation is recorded, a maximum value of the maximum representation values Aₘₐₓ in the m pre-evaluations is taken as a reference value A_{ref} of the evaluation for the subject, the representation of the amplitude features of the SSVEP obtained from a formal evaluation is Aₓ, and then an instantaneous evaluation score for the attention of the subject is:

$$A T = \frac{{A}_{x}}{{A}_{r e f}} \times 100 .$$

A final score of each evaluation for the subject is Score=f (AT, t), which is a distribution feature of the instantaneous evaluation score AT for the attention of the subject over time t. By means of several representation forms of Aₓ shown in FIG. 3, the final score is calculated through such distribution features, so that the Score can more objectively reflect the level of attention of the subject, and the evaluation can be more accurate and impartial.

As an implementation mode,

Score_{Δt}=∫ₜ_{₀}^{t}^{₀}^{+Δt}ATₓ/Δt

where to is a timing start time, and Δt is a time interval.

In addition, short-range (5 seconds) average attention score [Score]_5, medium-range (20 seconds) average attention score [Score]_20, long-range (60 seconds) average attention score [Score]_60, and so on may also be given respectively, and the value of Δt is taken as needed for a short range, a medium range and a long range, so as to make a more comprehensive evaluation on the evaluation results.

As other implementation modes, the Score may also be calculated by statistical methods such as mean and standard deviation.

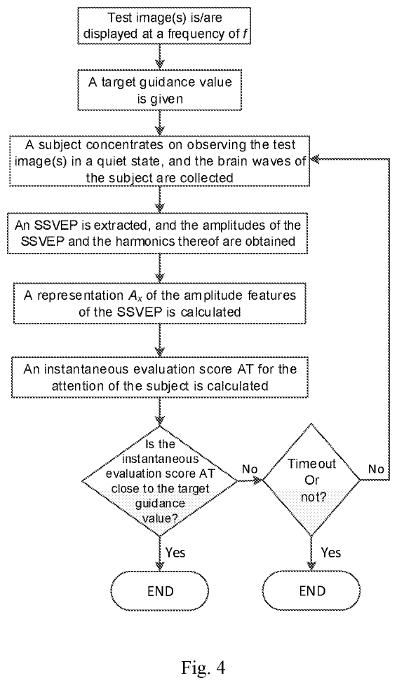

Based on the SSVEP-based attention evaluation method, the present invention provides an SSVEP-based attention training method, as shown in FIG. 4, including the following steps. Test image(s) is/are displayed at a frequency f, the vision of a trainee is stimulated, and the brain waves of the trainee are collected. An SSVEP related to the frequency f in the brain waves is extracted. A representation of the amplitude features of the SSVEP is used as an instantaneous evaluation result of the attention of the trainee. The instantaneous evaluation result is fed back to the trainee in real time, so that the trainee adjusts the attention according to the instantaneous evaluation result in real time.

In order to enable the trainee to better adjust the own attention, during the training process, the instantaneous evaluation result is fed back to the trainee in real time during the training process, and a target guidance value at each moment is given at the same time, so that the trainee performs attention adjustment according to the real-time instantaneous evaluation result and the target guidance value. Preferably, the degree of coincidence of the attention change of the trainee with the dynamically changing target guidance value is displayed in real time in the training process. Preferably, the trainee adjusts the attention according to the target guidance value, and when an error between the instantaneous evaluation result of the attention of the trainee and the target guidance value falls within 5%, the target guidance value is maintained for a period of time t and then changed.

As another implementation mode, the display brightness of the test image(s) is increased or decreased in the training, the trainee is guided to perform the attention adjustment training, and the change of brightness is ended when the instantaneous evaluation result returns to the level before the change of brightness or exceeds a set duration. Preferably, the test image(s) include(s) a plurality of target images, and at least one of display frequency, color and brightness attributes of the plurality of target images is different.

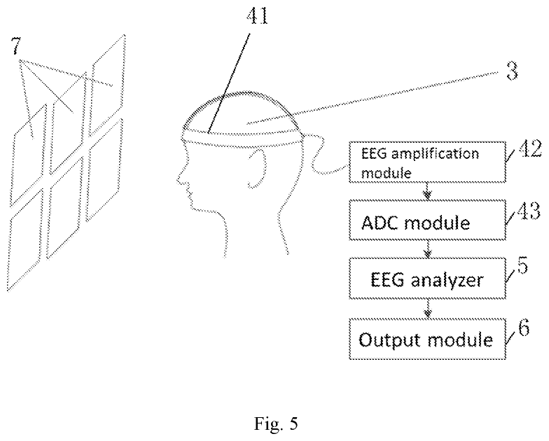

An SSVEP-based BCI for implementing the evaluation method and the training method, as shown in FIG. 5, includes an EEG collection device 4 and a visual evoking device for displaying an SSVEP-evoked image array 7. The EEG collection device 4 is worn on the head of a user. The SSVEP-based BCI further includes an EEG analyzer 5. The EEG analyzer 5 is connected to the EEG collection device 4, and is configured to extract an SSVEP related to a display frequency from the brain waves collected by the EEG collection device 4, obtain a representation Aₓ of the amplitude features of the SSVEP corresponding to each display frequency, and use a maximum value in the representation and the corresponding frequency as the output of the BCI. An EEG output module 6 is connected to the EEG analyzer 5 for outputting results obtained from the EEG analyzer 5 to other specified devices in a wireless/wired manner.

The EEG collection device 4 includes a headband 41 with electrodes, an EEG amplification module 42 and an analogue-to-digital conversion module 43.

As one of the implementation modes, a lower threshold of the representation value of the amplitude features of the SSVEP is preset, and when the representation value of the amplitude features of the SSVEP is lower than the lower threshold, the EEG analyzer outputs a stop signal or a zero value.

As one of the implementation modes, the EEG analyzer 5 records the amplitude of each display frequency, the amplitude of multiple frequency, and a duration for which these amplitudes exceed the lower threshold of the representation value of the amplitude features, as shown in FIG. 3, while acquiring the representation of the amplitude features of the SSVEP.

As one of the implementation modes, the EEG analyzer distinguishes attention targets of users according to the display frequency corresponding to the maximum value in the representation of the amplitude features of the SSVEP, and outputs the attention targets while outputting the maximum value of the representation of the amplitude features.

As one of the implementation modes, an upper threshold of the representation value of the amplitude features of the SSVEP is preset, and when the representation value of the amplitude features of the SSVEP is higher than the upper threshold, the EEG analyzer outputs the upper threshold.

The EEG analyzer records a duration for which the instantaneous representation value of the amplitude features of the SSVEP is greater than the lower threshold, the duration is divided into a short duration and a long duration according to a preset duration threshold, and triggering operations corresponding to the short duration and the long duration are output. For example, the short duration is taken as target determination, and the long duration is taken as target selection cancellation.

Application of an SSVEP-based BCI for controlling the motion of a controlled object is provided. A relationship between a running speed Vₓ of the controlled object and an SSVEP-based amplitude representation value Aₓ satisfies:

$${V}_{x} = \{ \begin{matrix}k \cdot {A}_{x} , & k \cdot {A}_{x} < {V}_{max} \\ {V}_{max} , & k \cdot {A}_{x} \geq {V}_{max}\end{matrix}$$

where k is a weighting coefficient, and Vₘₐₓ is the highest speed at which the controlled object runs.

As one of the implementation modes, the weight coefficient k is calculated by the following formula:

$$k = \frac{{V}_{max}}{2 \cdot {A}_{m e a n}}$$

where Aₘₑₐₙ is a mean value of the amplitude representation values of the SSVEP of users over a period of time.

With respect to the existing technology, in the SSVEP-based BCI, it is difficult to output a value that has a practical reference meaning and can reflect the continuous change of the degree of attention of users to targets, and only a yes/no judgment value can be output. The present invention, using continuously changing values as evaluation results of the attention level, has the beneficial effect of significantly expanding the field of application of the BCI, and brings the development of the BCI into a new stage.

In a specific attention evaluation example, the type and configuration of the EEG collection device, the closeness of the brain electrodes to the scalp, and the amplification and processing of the brain waves have become constant. At this time, by using the above technical means, the current value of the attention of a testee or subject can be obtained, and in combination with the attention benchmark obtained by the pre-evaluation result, the specific value of each change of attention has the reference significance of adapting to individual differentiation in the process of test or training in the form of percentage. The evaluation result of the attention is no longer a relative quantity, but an absolute quantity in the sense of a percentage value relative to the own attention benchmark of the testee or subject. Different individuals are comparable in the degree of attention by the percentage.

It will be appreciated by those of ordinary skill in the art that the foregoing description is by way of example only and is not intended to limit the invention so that, although the invention has been described in detail with reference to the foregoing examples, it will be understood by those skilled in the art that the technical solutions described in the foregoing examples are modified or some technical features are equivalently replaced. Any modifications, equivalent replacements and the like made within the spirit and principle of the present invention should fall within the scope of protection of the present invention.

1. An SSVEP-based attention evaluation method, comprising the following steps:
displaying test image(s) at a frequency f, stimulating the vision of a subject, and collecting the brain waves of the subject;
extracting an SSVEP related to the frequency f in the brain waves; and
using a representation Aₓ of the amplitude features of the SSVEP to evaluate the degree of attention of the subject, wherein Aₓ is a combination of the amplitude of the first-harmonic/fundamental frequency f and/or the amplitude of multiple frequency.

2. The SSVEP-based attention evaluation method according to claim 1, wherein a specific calculation formula of Aₓ is as follows:
$${A}_{x} = {\sum }_{i = 1}^{n} {a}_{i} \cdot {M}_{{f}_{i}}$$
where aᵢ is a weighting coefficient, and M_{fi} represents a function of the amplitude of the i^{th} harmonic component in the SSVEP.

3. The SSVEP-based attention evaluation method according to claim 1, wherein a calculation formula of the weighting coefficient aᵢ is as follows:
$${a}_{i} = \{ \begin{matrix}1 , & i is an odd number , & i < 6 \\ 0 , & i is an even number , & i < 6\end{matrix}$$

4. The SSVEP-based attention evaluation method according to claim 1, wherein before an attention evaluation for a new subject, m pre-evaluations are performed, a maximum representation value Aₘₐₓ of the amplitude features of the SSVEP in each pre-evaluation is recorded, a maximum value of the maximum representation values Aₘₐₓ in the m pre-evaluations is taken as a reference value A_{ref} of the evaluation for the subject, the representation of the amplitude features of the SSVEP obtained from a formal evaluation is Aₓ, and then an instantaneous evaluation score for the attention of the subject is:
$$A T = \frac{{A}_{x}}{{A}_{r e f}} \times 100 .$$

5. The SSVEP-based attention evaluation method according to claim 4, wherein a final score of each evaluation for the subject is Score=f (AT, t), which is a distribution feature of the instantaneous evaluation score AT for the attention of the subject over time t.

6. The SSVEP-based attention evaluation method according to claim 4, wherein the Score is divided into a short-range average attention score, a medium-range average attention score and a long-range average attention score, and a specific calculation formula is as follows:
Score_{Δt}=∫ₜ_{₀}^{t}^{₀}^{+Δt}ATₓ/Δt
where t₀ is a timing start time, Δt is a time interval, and Δt has different values for a short range, a medium range and a long range.

7. An SSVEP-based attention training method, comprising the following steps:
displaying test image(s) at a frequency f, stimulating the vision of a trainee, and collecting the brain waves of the trainee;
extracting an SSVEP related to the frequency fin the brain waves;
using a representation of the amplitude features of the SSVEP as an instantaneous evaluation result of the attention of the trainee; and
feeding back the instantaneous evaluation result to the trainee in real time, so that the trainee adjusts the attention according to the instantaneous evaluation result in real time.

8. The SSVEP-based attention training method according to claim 7, wherein the instantaneous evaluation result is fed back to the trainee in real time during the training process, and a target guidance value at each moment is given at the same time, so that the trainee performs attention adjustment according to the real-time instantaneous evaluation result and the target guidance value.

9. The SSVEP-based attention training method according to claim 7, further comprising: an attention adjustment training, specifically comprising:
increasing or decreasing the display brightness of the test image(s) in the training, guiding the trainee to perform the attention adjustment training, and ending the change of brightness when the instantaneous evaluation result returns to the level before the change of brightness or exceeds a set duration.

10. The SSVEP-based attention training method according to claim 7, wherein the test image(s) comprise(s) a plurality of target images, and at least one of display frequency, color and brightness attributes of the plurality of target images is different.

11. The SSVEP-based attention training method according to claim 8, wherein the degree of coincidence of the attention change of the trainee with the dynamically changing target guidance value is displayed in real time in the training process.

12. The SSVEP-based attention training method according to claim 9, wherein the trainee adjusts the attention according to the target guidance value, and when an error between the instantaneous evaluation result of the attention of the trainee and the target guidance value falls within 5%, the target guidance value is maintained for a period of time t and then changed.

13. An SSVEP-based Brain-Computer Interface (BCI), comprising a visual evoking device for displaying an SSVEP-evoked image, and an EEG collection device worn on the head of a user, the BCI being configured to implement the method of any one of the preceding claims, further comprising:
an EEG analyzer, wherein the EEG analyzer is connected to the EEG collection device, and is configured to extract an SSVEP related to a display frequency from the brain waves collected by the EEG collection device, obtain a representation Ax of the amplitude features of the SSVEP corresponding to each display frequency, and use a maximum value in the representation and the corresponding frequency as the output of the BCI.

14. The SSVEP-based BCI according to claim 13, wherein the EEG analyzer distinguishes attention targets of users according to the display frequency corresponding to the maximum value in the representation of the amplitude features of the SSVEP, distinguishes the attention targets by the size of the representation of the amplitude features of the SSVEP if the display frequencies of the target images are the same, and outputs the attention targets.

15. The SSVEP-based BCI according to claim 13, wherein
a lower threshold of the representation value of the amplitude features of the SSVEP is preset, and when the representation value of the amplitude features of the SSVEP is lower than the lower threshold, the EEG analyzer outputs a stop signal or a zero value, and
an upper threshold of the representation value of the amplitude features of the SSVEP is preset, and when the representation value of the amplitude features of the SSVEP is higher than the upper threshold, the EEG analyzer outputs the upper threshold.

16. The SSVEP-based BCI according to claim 15, wherein
the EEG analyzer records and outputs a duration for which the instantaneous representation value of the amplitude features of the SSVEP is greater than the lower threshold, the duration is divided into a short duration and a long duration according to a preset duration threshold, and triggering operations corresponding to the short duration and the long duration are output.

17. The SSVEP-based BCI according to claim 13, wherein a relationship between a running speed Vₓ of the controlled object and an SSVEP-based amplitude representation value Aₓ satisfies:
$${V}_{x} = \{ \begin{matrix}k \cdot {A}_{x} , & k \cdot {A}_{x} < {V}_{max} \\ {V}_{max} , & k \cdot {A}_{x} \geq {V}_{max}\end{matrix}$$
where k is a weighting coefficient, and Vₘₐₓ is the highest speed at which the controlled object runs.

18. The SSVEP-based BCI according to claim 17, wherein the weight coefficient k is calculated by the following formula:
$$k = \frac{{V}_{max}}{2 \cdot {A}_{m e a n}}$$
where Aₘₑₐₙ is a mean value of the amplitude representation values of the SSVEP of users over a period of time.
